(19) United States
(12) Patent Application Publication
(10) Pub. No.: US 2022/0125494 A1
(43) Pub. Date: Apr. 28, 2022

(54) CONNECTABLE LOCKING PLATE ASSEMBLY

(71) Applicant: INGROWTH BIOTECH CO., LTD., Kaohsiung City (TW)

(72) Inventors: Chih-Yu CHEN, Kaohsiung City (TW); Po-Han CHU, Kaohsiung City (TW); Royal SU, Kaohsiung City (TW); Jason SUN, Kaohsiung City (TW)

(73) Assignee: INGROWTH BIOTECH CO., LTD., Kaohsiung City (TW)

(21) Appl. No.: 17/401,685

(22) Filed: Aug. 13, 2021

(30) Foreign Application Priority Data
Oct. 26, 2020 (CN) 202011157235.3

(51) Int. Cl. A61B 17/80

(52) U.S. Cl. CPC A61B 17/8057; A61B 17/8066

(57) ABSTRACT

A connectable locking plate assembly comprises a locking plate unit, a connection unit, and a locking component. The locking plate unit comprises multiple interconnecting fixing parts. The connection unit comprises multiple interconnecting positioning parts, and at least one of the positioning parts has an assembly recess. One of the fixing parts is inserted into the assembly recess. The locking component is mounted through the screw connecting hole on the connection unit and is screwed into the screw setting hole of the locking plate unit. Therefore, the connectable locking plate assembly can be assembled into an appropriate structure by assembling the locking plate unit, the connection unit, and the locking component to meet the sizes and outer shapes of the bones at the fracture site. Therefore, the assembly can increase the fitting and fixing effect to the bones, and can enhance convenience during assembling.

### BACKGROUND OF THE INVENTION

### 1. Field of the Invention

The present invention relates to a connectable locking plate assembly, especially to a connectable locking plate assembly for connecting locking plates and can support and fix bones.

### 2. Description of the Prior Arts

Bones in the human body not only have functions of supporting the torso and protecting vital organs, but also can cooperate with muscles to aid the human body with various movements and exercising, which makes the bones indispensable in the human body. Even though the bones are solid and can provide functions of supporting the torso and protecting the vital organs of the human body, they could still fracture because of internal diseases (such as Osteoporosis, Osteogenesis Imperfecta, etc.) or strong impact.

Since bones need longer natural healing time, if they are not fixed rigidly during the natural healing, sequelae such as wound infection, malunion, muscle atrophy, joint dysfunction, etc. might be caused. Therefore, when treating bone fracture nowadays, the fractured portion or the splinters are restored to the original position, and the bones are fixed rigidly during natural healing, to prevent sequelae from happening.

In order to fix the fractured bone rigidly, mostly the treatment is to set a conventional locking plate at the fracture site of the bone to assure the bone's position. The conventional locking plate used nowadays is an integrated component. Though the conventional locking plates are manufactured into various preset sizes and with specifications, the bone sizes or the curvatures of bone surfaces differ from patient to patient. Therefore, when using those conventional locking plates, it is necessary to cut or reshape the conventional locking plates according to the actual bone size and surface shapes of the patient to be treated before fitting the conventional locking plate to the bone surface. If the bone size of the patient differs too much from the conventional locking plate, customizing a locking plate via the manufacturing company may even be necessary. Thus, it is hard for the conventional locking plates to have customized design for different patients and therefore the conventional locking plates are not convenient for use.

### SUMMARY OF THE INVENTION

The technical problem that the present invention wants to solve is that the conventional locking plate is hard to be customized for different patients and thus is less convenient for use.

A connectable locking plate assembly of the present invention comprises at least one locking plate unit, at least one connection unit, and at least one locking component. The at least one locking plate unit has multiple fixing parts connected to each other, and each one of the fixing parts has a screw setting hole. The at least one connection unit has multiple positioning parts connected to each other, and each one of the positioning parts has a screw connecting hole. At least one of the positioning parts has an assembly recess connecting to the screw connecting hole thereof. One of the fixing parts of the at least one locking plate unit is inserted into the assembly recess. The at least one locking component is mounted through the screw connecting hole of the at least one connection unit and is screwed in the screw setting hole of the at least one locking plate unit.

The connectable locking plate assembly comprises two connection units and at least one locking component. The two connection units are connected to each other, and each one of the connection units has multiple positioning parts connected to each other. Each one of the positioning parts has a screw connecting hole. At least one of the positioning parts has an assembly recess connecting to the screw connecting hole thereof. One of the positioning parts of the other connection unit is inserted into the assembly recess. The at least one locking component is mounted through the screw connecting hole of the connection unit, which is inserted into the assembly recess of the other connection unit, and the at least one locking component is screwed into the screw connecting hole of the connection unit, which is inserted by the other connection unit.

To use the present invention, pair the assembly recess of the connection unit with the locking component to connect the locking plate unit or another connection unit, thus facilitating medical personnel to match various configurations of the connectable locking plate assemblies through connecting the locking plate units or the connection units of different shapes. This effectively increases the diversity of the connectable locking plate assembly, and also the medical personnel can assemble the appropriate connectable locking plate assembly in accordance with the sizes and outer shapes of the bones at the fracture site only through connecting or combining the locking plate unit and the connection unit. Besides, the medical personnel can complete the fixing of the connection unit or the locking plate unit only through the locking component, further to enhance the convenience while assembling.

### BRIEF DESCRIPTION OF THE DRAWINGS

FIG. 1 is an exploded perspective view of the first connecting configuration of a connectable locking plate assembly in accordance with the present invention;

FIG. 2 is a perspective view of the first connecting configuration of the connectable locking plate assembly in FIG. 1;

FIG. 3 is a partial sectional view of the connectable locking plate assembly in FIG. 1;

FIG. 4 is a perspective view of the second connecting configuration of the connectable locking plate assembly;

FIG. 5 is a perspective view of the third connecting configuration of the connectable locking plate assembly;

FIG. 6 is a perspective view of the fourth connecting configuration of the connectable locking plate assembly;

FIG. 7 is an exploded perspective view of the fourth connecting configuration of the connectable locking plate assembly in FIG. 6;

FIG. 8 is a perspective view of the fifth connecting configuration of the connectable locking plate assembly;

FIG. 9 is a perspective view of the connectable locking plate assembly, showing a pivot connection between a connection unit and a locking plate unit or a pivot connection between two connection units;

FIG. 10 is an operational perspective view of a first application of the connectable locking plate assembly; and

FIG. 11 is an operational perspective view of a second application of the connectable locking plate assembly.

### DETAILED DESCRIPTION OF THE PREFERRED EMBODIMENTS

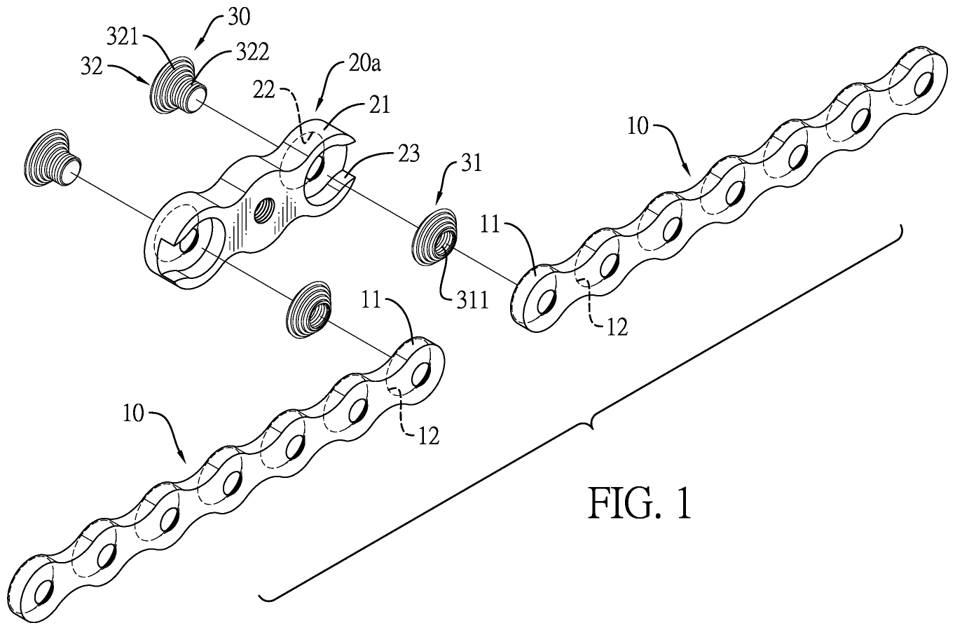

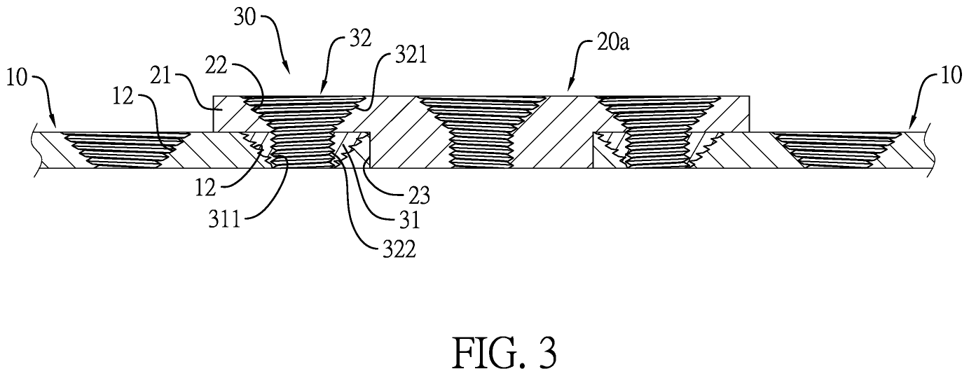

FIGS. 1 to 5 are various connecting configurations of a connectable locking plate assembly in accordance with the present invention. The connectable locking plate assembly comprises at least one locking plate unit 10, at least one connection unit 20a˜20c, and at least one locking component 30.

With reference to FIGS. 1 to 5, the locking plate unit 10 comprises multiple fixing parts 11 connected to each other, and each one of the fixing parts 11 has a screw setting hole 12.

With reference to FIGS. 1 to 5, the connection unit 20a˜20c comprises multiple positioning parts 21 connected to each other, and each one of the positioning parts 21 has a screw connecting hole 22. At least one of the positioning parts 21 has an assembly recess 23 connecting to the screw connecting hole 22 thereof. The fixing part 11 of the locking plate unit 10 can be inserted into the assembly recess 23.

With reference to FIGS. 1 to 5, the locking component 30 is mounted through the screw connecting hole 22 of the connection unit 20a˜20c and screwed in the screw setting hole 12 of the locking plate unit 10.

The connectable locking plate assembly comprises multiple connection units 20a˜20c and multiple locking components 30. The assembly recess 23 of the connection unit 20a˜20c can provide the fixing part 11 of the locking plate unit 10 or the positioning part 21 of another connection unit 20a˜20c to be inserted therein. The locking component 30 is mounted through the screw connecting hole 22 of the connection unit 20a˜20c and then screwed in the screw setting hole 12 of the locking plate unit 10 or the screw connecting hole 22 of another connection unit 20a˜20c.

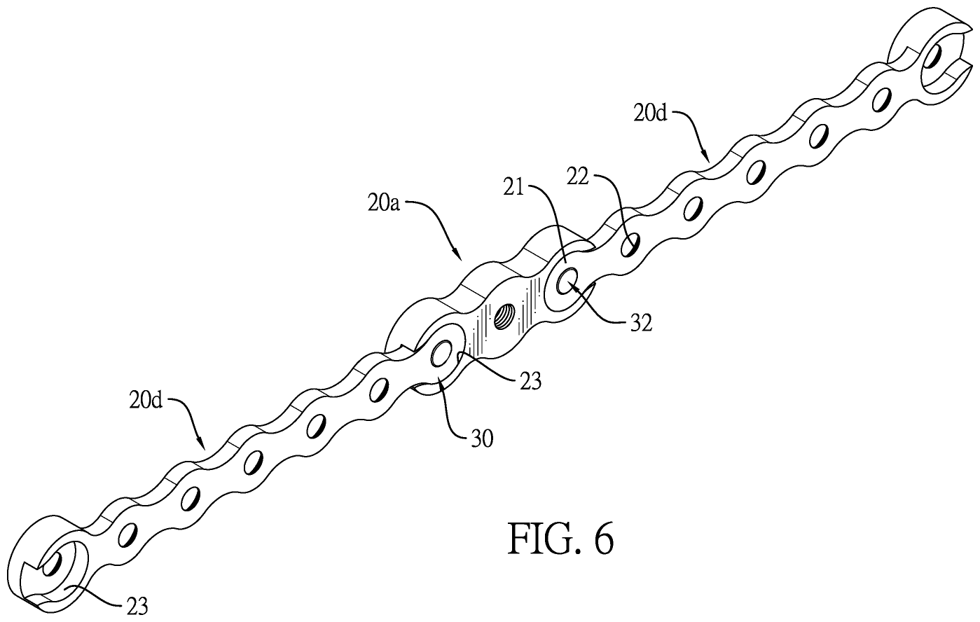

FIGS. 6 to 8 are the other two configurations of the connectable locking plate assembly of the present invention, comprising multiple connection units 20a, 20d and at least one locking component 30.

With reference to FIGS. 6 to 8, the multiple connection units 20a, 20d can be connected to each other. Each one of the connection units 20a, 20d comprises multiple positioning parts 21 connected to each other, and each one of the positioning parts 21 has a screw connecting hole 22. At least one of the positioning parts 21 has an assembly recess 23 connecting to the screw connecting hole 22 thereof. The assembly recess 23 can provide the positioning part 21 of the connection unit 20a, 20d to be inserted therein.

With reference to FIGS. 6 to 8, the locking component 30 is mounted through the screw connecting hole 22 of the connection unit 20a, 20d and screwed in the screw connecting hole 22 of another connection unit 20a, 20d.

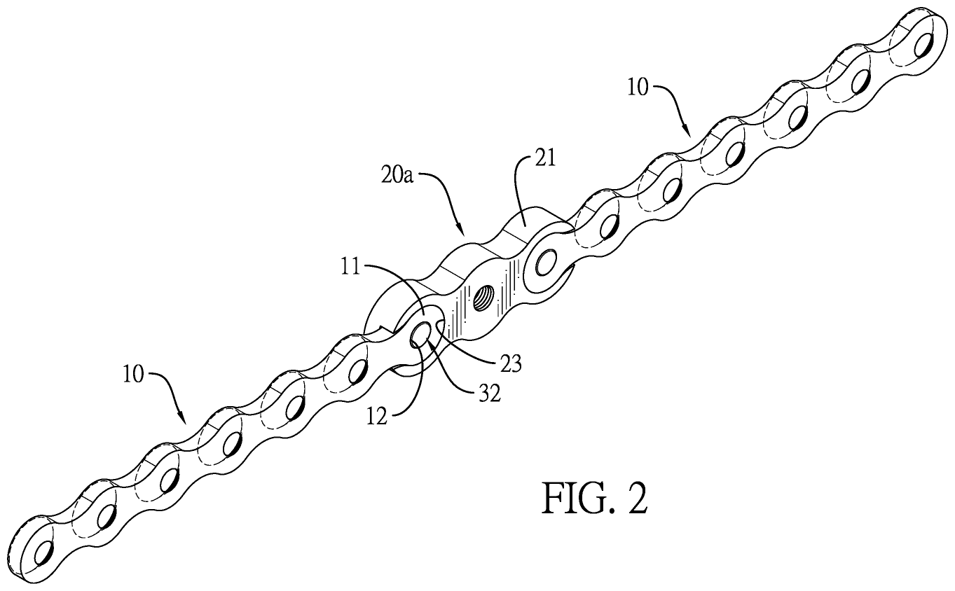

Also, with reference to FIGS. 2, 3 and 7, the screw setting hole 12 of the locking plate unit 10 and the screw connecting hole 22 of the connection unit 20a˜20d are tapper screw holes. The locking component 30 comprises a tapper bush 31 and a locking screw 32. An outer surface of the tapper bush 31 is formed with outer threads corresponding to the screw setting hole 12 of the locking plate unit 10 and the screw connecting hole 22 of the connection unit 20a˜20d. The tapper bush 31 is formed with an inner screw hole 311. The tapper bush 31 can be threaded in the screw setting hole 12 of the locking plate unit 10 or the screw connecting hole 22 of the connection unit 20a˜20d. The locking screw 32 is threaded in the screw connecting hole 22 of the connection unit 20a˜20d and the inner screw hole 311 of the tapper bush 31.

Besides, the locking screw 32 comprises a head part 321 and a bolt part 322. One end of the head part 321 is formed with a tool recess, and the tool recess may be slotted, cruciform, hexalobular, hexagonal, etc. The outer surface of the head part 321 is formed with outer threads corresponding to the screw setting hole 12 of the locking plate unit 10 and the screw connecting hole 22 of the connection unit 20a˜20d. The bolt part 322 is formed on the end, which is opposite to the tool recess, of the head part 321. The head part 321 of the locking screw 32 can be threaded in the screw setting hole 12 of the locking plate unit 10 or the screw connecting hole 22 of the connection unit 20a˜20c. The bolt part 322 can be threaded to the inner screw hole 311 of the tapper bush 31.

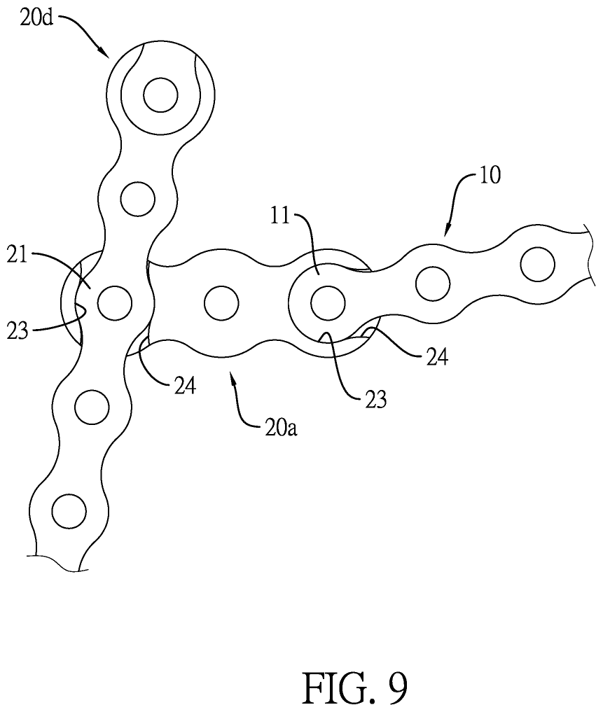

In addition, with reference to FIG. 9, the assembly recess 23 of the connection unit 20a is formed with at least one opening 24 on its side surface. The fixing part 11 of the locking plate unit 10 or the positioning part 21 of another connection unit 20d can be inserted into the assembly recess 23 through the opening 24. An inner diameter of the opening 24 is larger than a width of a junction between the fixing parts 11 of the locking plate units 10 or a width of a junction between the positioning parts 21 of another connection unit 20d.

The connectable locking plate assembly in accordance with the present invention can be connected to the locking plate unit 10 or another connection unit 20a˜20d through pairing the assembly recess 23 of the connection unit 20a˜20d with the locking component 30. When using the connection unit 20a˜20d to connect the locking plate unit 10 or another connection unit 20a˜20d, the tapper bush 31 of the locking component 30 is mounted in the screw setting hole 12 of the fixing part 11 of the locking plate unit 10 or in the screw connecting hole 22 of the positioning part 21, and then the fixing part 11 or the positioning part 21 that has been mounted with the tapper bush 31 is inserted into the assembly recess 23 of the connection unit 20a˜20d. Afterwards, the locking screw 32 of the locking component 30 is threaded in the screw connecting hole 22 of the connection unit 20a˜20d and the inner screw hole 311 of the tapper bush 31, thereby achieving the functions of connecting and fixing.

Therefore, medical personnel can assemble the appropriate connectable locking plate assembly in accordance with the sizes and outer shapes of the bones at the fracture site, through connecting or combining the locking plate unit 10 and the connection unit 20a˜20d. Connecting configurations of the connectable locking plate assembly are elaborated as follows.

With reference to FIGS. 1 to 3, in the first connecting configuration of the present invention, the connectable locking plate assembly comprises two locking plate units 10, a connection unit 20a, and two locking components 30. The positioning parts 21 at the two opposite ends of connection unit 20a respectively have the assembly recess 23. The two fixing parts 11 at an end of the two locking plate units 10 can cooperate with the two locking components 30 to be connected to the assembly recesses 23 at the two ends of connection unit 20a.

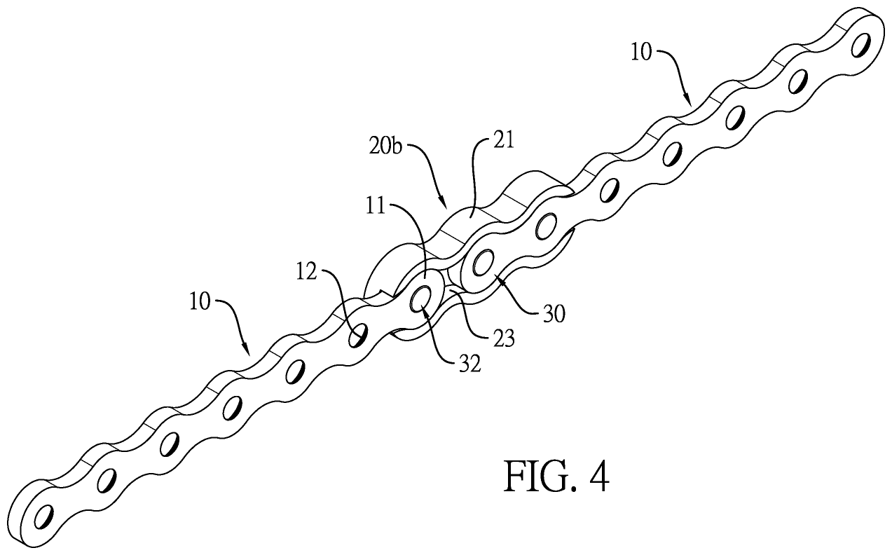

With reference to FIG. 4, in the second connecting configuration of the present invention, the connectable locking plate assembly comprises two locking plate units 10, a connection unit 20b and three locking components 30. The positioning parts 21 of the connection unit 20b all have the assembly recesses 23, and the assembly recesses 23 of the positioning parts 21 of the connection unit 20b are connected to each other. The fixing parts 11 of the ends of the two locking plate units 10 can cooperate with the three locking components 30 to be connected to the assembly recesses 23 of the connection unit 20b.

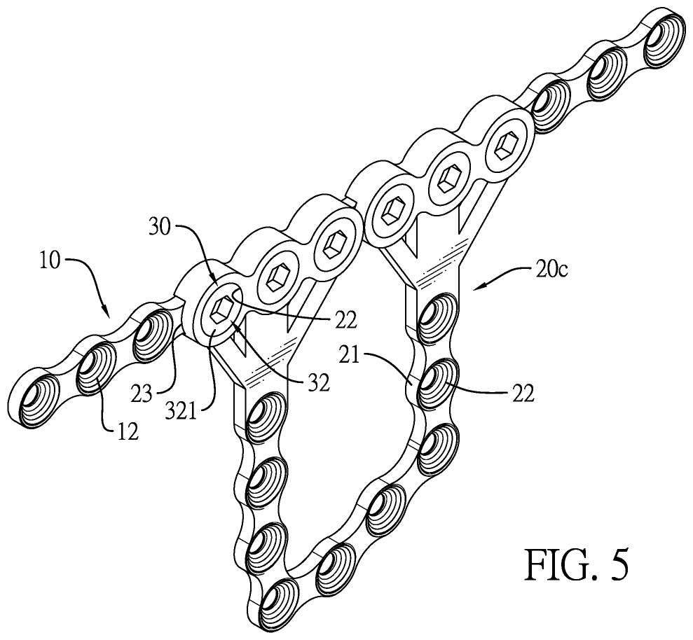

With reference to FIG. 5, in the third connecting configuration of the present invention, the connectable locking plate assembly comprises a locking plate unit 10, a connection unit 20c, and multiple locking components 30. The connection unit 20c can be U-shaped, and the two ends of the connection unit 20c are respectively formed with multiple assembly recesses 23 connected to each other. In other words, the adjacent positioning parts 21 at one of the two ends of the connection unit 20c all have the assembly recesses 23, and those assembly recesses 23 communicate with each other. Also, the adjacent positioning parts 21 at the other end of the connection unit 20c all have the assembly recesses 23, and those assembly recesses 23 communicate with each other. The assembly recesses 23 at the two ends of the connection unit 20c can cooperate with the multiple locking components 30 to be connected to the fixing parts 11 of the locking plate unit 10.

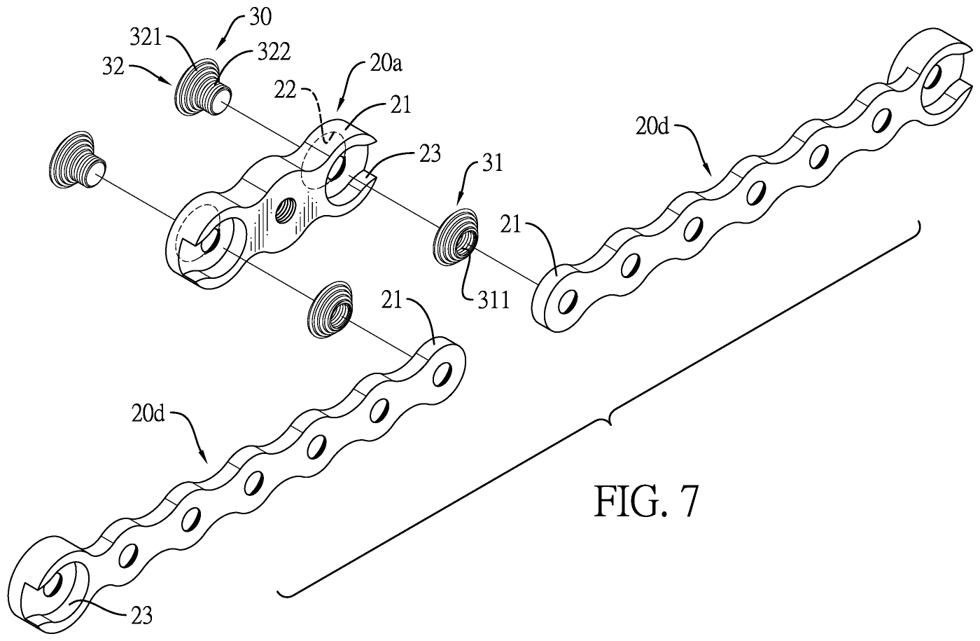

Besides, with reference to FIGS. 6 and 7, in the fourth connecting configuration of the present invention, the connectable locking plate assembly comprises three connection units 20a, 20d and two locking components 30. The positioning parts 21 at the two opposite ends of one of the three connection units 20a, 20d have the assembly recesses 23 respectively. The assembly recesses 23 at the connection unit 20a can cooperate with the two locking components 30 to be connected to the two positioning parts 21 of the other two connection units 20d.

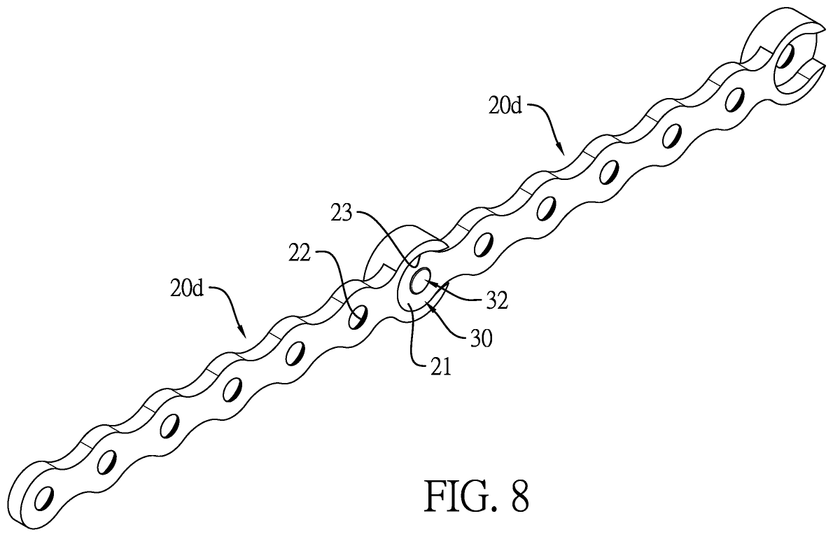

Also, with reference to FIG. 8, in the fifth connecting configuration of the present invention, the connectable locking plate assembly comprises two connection units 20d and a locking component 30. The positioning parts 21 at one end of each of the two connection units 20d have the assembly recesses 23. The assembly recess 23 at one of the two connection units 20d can cooperate with the locking component 30 to be connected to the positioning parts 21 of the other connection unit 20d.

In addition, with reference to FIG. 9, because the inner diameter of the opening 24 is larger than the width of the junction between the fixing parts 11 of the locking plate unit 10 or the width of the junction between the positioning parts 21 of the connection unit 20d, when the fixing part 11 of the locking plate unit 10 or the positioning part 21 of another connection unit 20d is inserted into the assembly recess 23 of the connection unit 20a, a pivot clearance is formed between the opening 24 and the junction which is between the fixing parts 11 of the locking plate units 10 or the junction which is between the positioning parts 21 of the connection unit 20d. Therefore, the locking plate unit 10 or another connection unit 20d can pivot relative to the connection unit 20a. Thus, medical personnel can pivot the locking plate unit 10 or another connection unit 20d relative to the connection unit 20a according to the patient's bone shape to achieve a better fixing effect.

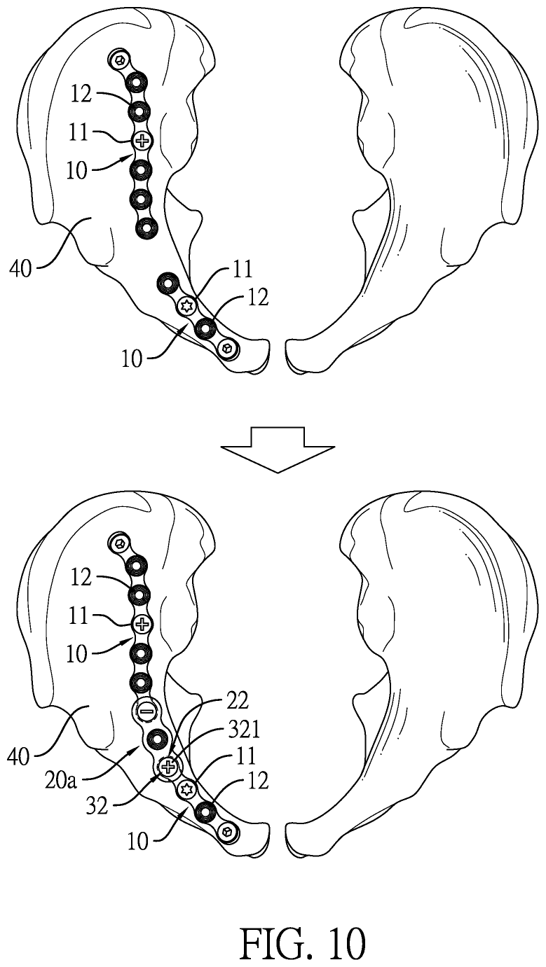

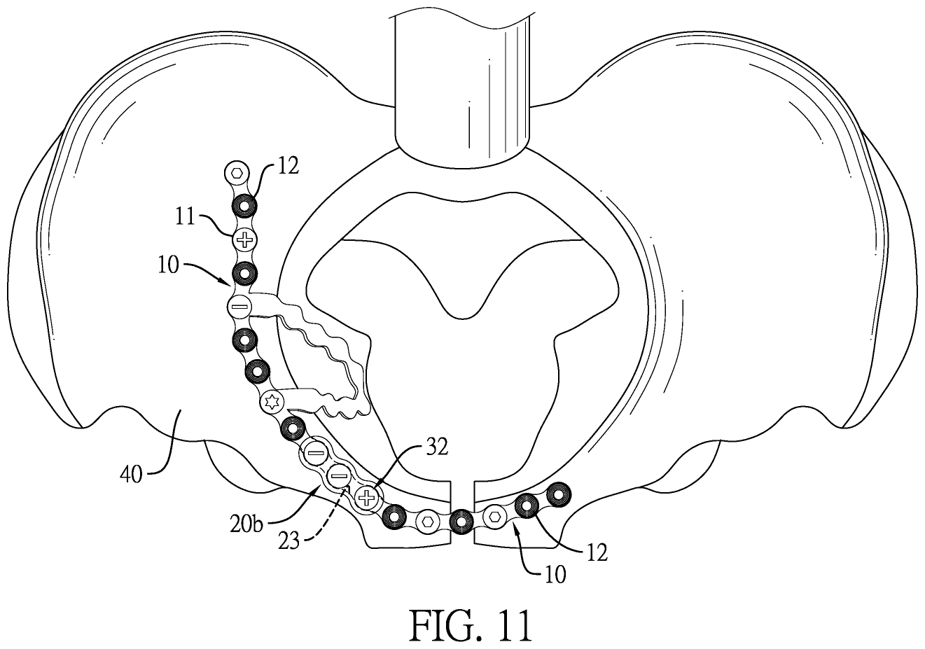

Therefore, with reference to FIG. 10, when using the connectable locking plate assembly, the medical personnel can respectively mount multiple locking plate units 10 at the fracture site and connect said multiple locking plate units 10 by the connection unit 20a and the locking component 30. With reference to FIG. 11, furthermore, the medical personnel also can change shapes of the locking plate unit 10 or the connection unit 20a˜20c in accordance with the bone sizes, the curvature of bone surfaces and the degree of injury of the patient, which not only makes the connectable locking plate assembly fit bones better and achieve higher fixing effect, but also increases applicability of the connectable locking plate assembly through different connecting configurations and at the same time improving convenience while assembling.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A connectable locking plate assembly comprising:
at least one locking plate unit each having
multiple fixing parts connected to each other, and each one of the fixing parts having;
a screw setting hole;
at least one connection unit each having
multiple positioning parts connected to each other, and each one of the positioning parts having
a screw connecting hole;
wherein at least one of the positioning parts has
an assembly recess connecting to the screw connecting hole thereof; one of the fixing parts of the at least one locking plate unit inserted into the assembly recess; and
at least one locking component mounted through the screw connecting hole of the at least one connection unit and screwed in the screw setting hole of the at least one locking plate unit.

2. The connectable locking plate assembly as claimed in claim 1, wherein
the at least one locking plate unit includes multiple said locking plate units;
the at least one locking component includes multiple said locking components;
the multiple locking plate units are respectively mounted on the multiple positioning parts of the at least one connection unit;
the multiple locking components are respectively mounted through the multiple screw connecting holes of the multiple positioning parts of the at least one connection unit, and the multiple locking components are respectively screwed in the multiple screw setting holes of the multiple locking plate units.

3. The connectable locking plate assembly as claimed in claim 1, wherein
the screw setting hole of the at least one locking plate unit and the screw connecting hole of the at least one connection unit are tapper screw holes;
the at least one locking component each comprises
a tapper bush having
outer threads formed on an outer surface of the tapper bush and corresponding to the screw setting hole of the at least one locking plate unit, such that the tapper bush is capable of being threaded in the screw setting hole of the at least one locking plate unit; and
an inner screw hole; and
a locking screw threaded in the screw connecting hole of the at least one connection unit and the inner screw hole of the tapper bush.

4. The connectable locking plate assembly as claimed in claim 2, wherein
the screw setting holes of the multiple locking plate units and the screw connecting hole of the at least one connection unit are tapper screw holes;
each one of the multiple locking components comprises
a tapper bush having
outer threads formed on an outer surface of the tapper bush and corresponding to the screw setting holes of the locking plate units, such that the tapper bush is capable of being threaded in the screw setting hole of one of the locking plate units; and
an inner screw hole; and
a locking screw threaded in the screw connecting hole of the at least one connection unit and the inner screw hole of the tapper bush.

5. The connectable locking plate assembly as claimed in claim 1, wherein in each of the at least one connection unit,
each of the positioning parts adjacent to each other at one of two ends of the connection unit has the assembly recess, and the assembly recesses in said positioning parts communicate with each other; and
each of the positioning parts adjacent each other at the other end of the connection unit has the assembly recess, and the assembly recesses in said positioning parts communicate with each other.

6. The connectable locking plate assembly as claimed in claim 1, wherein the assembly recess has
at least one opening on a side wall of the assembly recess; an inner diameter of the at least one opening being larger than a width of a junction between two adjacent ones of the fixing parts of the at least one locking plate unit, such that each one of the fixing parts of the at least one locking plate unit is capable of being inserted into the assembly recess through the at least one opening.

7. A connectable locking plate assembly comprising:
two connection units connected to each other, and each one of the connection units having
multiple positioning parts connecting to each other, and each one of the positioning parts having
a screw connecting hole;
wherein at least one of the positioning parts has
an assembly recess connecting to the screw connecting hole thereof; one of the positioning parts of the other connection unit inserted into the assembly recess; and
at least one locking component mounted through the screw connecting hole of the connection unit, which is inserted into the assembly recess of the other connection unit, and the at least one locking component screwed into the screw connecting hole of the connection unit, which is inserted by the other connection unit.

8. The connectable locking plate assembly as claimed in claim 7, wherein
the screw connecting holes of the connection units are tapper screw holes;
the at least one locking component each comprises
a tapper bush having
outer threads formed on an outer surface of the tapper bush and corresponding to the screw connecting holes of the connection units, such that the tapper bush is capable of being threaded in the screw connecting hole of one of the connection units; and
an inner screw hole; and
a locking screw threaded in the screw connecting hole of one of the connection units, which is inserted by the other connection unit, and in the inner screw hole of the tapper bush in one of the connection units, which is inserted into the assembly recess of the other connection unit.

9. The connectable locking plate assembly as claimed in claim 8, wherein in each one of the connection units,
each of the positioning parts adjacent to each other at one of two ends of said connection unit has the assembly recess, and the assembly recesses in said positioning parts communicate with each other; and
each of the positioning parts adjacent to each other at the other end of said connection unit has the assembly recess, and the assembly recesses in said positioning parts communicate with each other.

10. The connectable locking plate assembly as claimed in claim 7, wherein the assembly recess has
at least one opening on a side wall of the assembly recess; an inner diameter of the at least one opening being larger than a width of a junction between two adjacent ones of the positioning parts of the connection units, such that each one of the positioning parts of the other connection unit is capable of being inserted into the assembly recess through the at least one opening.
